(19) United States
(12) Patent Application Publication
(10) Pub. No.: US 2012/0071690 A1
(43) Pub. Date: Mar. 22, 2012

(54) PRODUCTION OF FORMAMIDES

(75) Inventors: Marek Pazicky, Heidelberg (DE); Thomas Schaub, Neustadt (DE); Ansgar Gereon Altenhoff, Heidelberg (DE); Donata Maria Fries, Mannheim (DE); Rocco Paciello, Bad Durkheim (DE)

(73) Assignee: BASF SE, Ludwigshafen (DE)

(21) Appl. No.: 13/234,549

(22) Filed: Sep. 16, 2011

Related U.S. Application Data
(60) Provisional application No. 61/383,742, filed on Sep. 17, 2010.

(51) Int. Cl. C07C 231/10

(52) U.S. Cl. 564/132

(57) ABSTRACT

A process is proposed for production of formamides by reaction of carbon dioxide with hydrogen in a hydrogenation reactor I in the presence of
a catalyst comprising an element from group 8, 9 or 10 of the periodic table,
a tertiary amine comprising at least 6 carbon atoms per molecule, and also
a polar solvent,
   to form formic acid-amine adducts as intermediates, which are subsequently reacted with ammonia or amines in a reactor to obtain a two-phase liquid reaction effluent from which the liquid phase enriched with the formamides is distillatively separated to recover the formamide.

### CROSS-REFERENCE TO RELATED APPLICATIONS

This Application claims the benefit of U.S. Provisional Application 61/383,742 filed on Sep. 17, 2010 which is incorporated by reference in its entirety.

### BACKGROUND OF THE INVENTION

This patent application claims the benefit of pending U.S. provisional patent application Ser. No. 61/383,742 filed on 17 Sep. 2010, incorporated in its entirety herein by reference.

The present invention relates to a process for production of formamides, i.e., formamide and its N-substituted derivatives, from carbon dioxide.

Formamide and its derivatives are important selective solvents and extractants because of their polarity. They are used to extract butadiene from C₄ cuts, acetylene from cracker C₂-fractions and aromatics from aliphatics, for example.

Large scale industrial manufacturing processes for formamide and its alkyl derivatives all have hitherto used carbon monoxide as C₁ building block:

Formamide, N-alkylformamides and N,N-dialkylformamides are obtained by reacting methyl formate with ammonia, N-alkylamines and N,N-dialkylamines, respectively. The methanol freed in the course of the reaction can be returned into the methyl formate synthesis from carbon monoxide and methanol.

A further synthesis used on an industrial scale reacts ammonia or the above-mentioned amines with carbon monoxide directly, instead of methyl formate, at 20 to 100° C. and 2 to 10 MPa. Methanol is used as a solvent and alkoxides are used as catalysts (Hans-Jürgen Arpe, Industrielle Organische Chemie, 6^{th} edition, 2007, pages 48 to 49).

Carbon monoxide is disadvantageous because of its toxicity, which necessitates strict safety precautions. Furthermore, the relatively high pressures involved in the production of formamides from carbon monoxide are cost intensive.

There has accordingly been no shortage of attempts to replace carbon monoxide by the inexpensive, nontoxic and widely available C₁ building block carbon dioxide (e.g. Applied Homogeneous Catalysis with Organometallic Compounds, volume 2, pages 1058 to 1072, 1996, VCH Verlagsgesellschaft and W. Leitner, Angew. Chem. Int. Ed. Engl. 1995, 34, pages 2207 to 2221).

There are numerous studies relating to the hydrogenation of carbon dioxide in the presence of primary or secondary amines to form ammonium formates and/or formamides by use of heterogeneous catalysts.

However, the activity of heterogeneous catalysts following their removal and recycling into the hydrogenation declines.

Therefore, homogeneous catalysts are preferred for the reaction.

U.S. Pat. No. 3,530,182 (Shell Oil Company, priority date Dec. 26, 1967) and Tetrahedron Letters No. 5, pages 365 to 368 (1970) show that homogeneous catalysts can also be used for the hydrogenation of carbon dioxide in the presence of primary and secondary amines to form formamides. Halogen-containing compounds of transition metals are suitable for example, particularly of the elements copper, zinc, cadmium, palladium and platinum. According to example 1, carbon dioxide is hydrogenated in the presence of dimethylamine and zinc bromide as catalyst at 155° C., 2.76 MPa of carbon dioxide and 2.76 MPa of hydrogen. After 17 hours dimethylformamide is obtained with a TOF value of 1.45. The TOF value (Turn Over Frequency) is a measure of the efficiency of the catalyst and indicates how many moles of product are formed per mole of catalyst per hour, cf. J. F. Hartwig: Organotransition Metal Chemistry, 1^{st} edition, 2010 University Science Books, Sausalito/California p. 545).

Carbon dioxide can also be hydrogenated in the presence of dimethylamine and homogeneously dissolved platinum complex compounds of the formula (μ-Pt₂(Ph₂P—CH₂—PPh₂)₃) to form dimethylformamide. TOF values of 57.3 are achieved at 100° C. and 11.4 MPa overall pressure (S. Schreiner et al. J. Chem. Soc., Chem. Commun., 1988, pages 602 to 603).

An enormous increase in TOF was achieved through the use of homogeneously dissolved ruthenium complex compounds comprising trialkylphosphine ligands. The hydrogenation of carbon dioxide was carried out in the presence of ammonia, primary or secondary amines and corresponding ammonium carbamates under supercritical conditions (reaction temperature >31° C., CO₂ pressure >7.4 MPa) in a single phase (P. G. Jessop, Y. Hsiao, T. Ikariya, R. Noyori, JACS 1996, 118, pages 351 to 352 and EP 0 652 202 A1, pages 5 to 6, 14 and Table 6, Research Development Corporation of Japan, priority dates Nov. 4, 1993 and Jun. 7, 1994). Tables 6 and 7 in JACS and table 6 in EP 0 652 202 A1 summarize the results. The highest TOF value was obtained after 70 hours at 6000 in the hydrogenation of carbon dioxide at 100° C., 13 MPa of carbon dioxide and 8 MPa of hydrogen in the presence of dimethylamine in the form of the ammonium carbamate. The catalyst used was RuCl₂[P(CH₃)₃]₄ (JACS, Table 7).

The preparation of formic acid by hydrogenation of carbon dioxide in the presence of the same or similar catalysts and sub- or supercritical conditions is carried out in the presence of tertiary amines which trap the formic acid in the form of adducts with the tertiary amines. In contrast thereto, amides are prepared in the absence of tertiary amines (JACS, pages 346, column 2, lines 3 to 10). It must be assumed that the formic acid initially formed is trapped by ammonia or the primary or secondary amines as a salt. This salt converts into formamides and water in a homogeneous phase at as low a temperature as 100° C. when supercritical conditions are imposed.

A further increase in the TOF values after 2 hours to 370,000 was obtained on using ruthenium complexes with bidentate phosphines as ligands (O. Kröcher, R. A. Köppel, A. Baiker, Chem. Comm. 1997, pages 453 to 454, Table 1).

EP 0 095 321 A, EP 0 151 510 A and EP 0 181 078 A (BP Chemicals Ltd.) disclose the hydrogenation of carbon dioxide in the presence of a homogeneous catalyst comprising a transition metal of transition group VIII (group 8, 9, 10), of a tertiary amine and of a polar solvent to form an adduct of formic acid and the tertiary amine. Ruthenium-based carbonyl, halide and/or triphenylphosphine-containing complex catalysts and rhodium-phosphine complexes are mentioned as preferred homogeneous catalysts. The hydrogenation is carried out at a carbon dioxide partial pressure of up to 6 MPa, a hydrogen partial pressure of up to 25 MPa and a temperature in the range from about room temperature to 200° C.

JP 11322687 reveals that the reaction of aliphatic aldehydes with formaldehyde in the presence of tertiary amines to form polyalcohols generates salts of formic acid and tertiary amines. These salts are hitherto inutile by-products. The application teaches reacting the salts at 20 to 130° C. with primary or secondary amines to form formamides, tertiary amines and water. The water of reaction is preferably removed by azeotropic distillation following addition of a solvent such as toluene for example. In the single exemplary embodiment, triethylammonium formate and toluene are mixed and admixed with piperidine (molar ratio of formate to piperidine=1:1.2). Under reflux, the toluene-water heteroazeotrope is separated off and the water removed from the system. The yield of N-formylpiperidine and triethylamine was quantitative.

It is known from Chemie Ingenieur Technik (75), pages 877 to 883 (2003) to react carbon dioxide with hydrogen in the presence of dimethylamine, toluene, water and rhodium- or ruthenium-phosphine complexes to form dimethylformamide. According to FIG. 5, TOF values of around 260 were obtained at 120° C., a partial pressure ratio of hydrogen to carbon dioxide of 1:1 (p_{cold}=5 MPa).

FIG. 7 shows an integrated processing concept for synthesis and workup of dimethylformamide: the reaction effluent consists of two liquid phases which are separated from each other. The top phase, which comprises toluene and catalyst, is concentrated and then returned into the synthesis stage. The bottom aqueous phase, which comprises the target product dimethylformamide, is extracted with toluene and worked up by distillation.

There are no indications as to which pressure is used to recycle excess hydrogen and excess carbon dioxide into which stage. Moreover, it is disadvantageous that marked losses of catalyst are observed despite extracting the aqueous phase with toluene. Furthermore, toluene is extraneous to the process and therefore has to be expensively and inconveniently removed before the extract is recycled into the hydrogenation stage.

The prior art shows that the hydrogenation of carbon dioxide in the presence of homogeneously dissolved ruthenium-phosphine complexes as catalysts and ammonia or primary or secondary amines gives amide TOF values which are attractive for industrial processes. However, none of the documents cited discloses how the hydrogenation effluents can be worked up in an economical manner to separate the homogeneous catalyst from the reaction products and return it into the hydrogenation stage (O. Kröcher, R. A. Köppel, A. Baiker, Chem. Commun. 1996, page 1497, left hand column, 1^{st} paragraph). Economical in this context means that it would be very advantageous if the carbon dioxide-hydrogen mixture, which is under high pressure during the hydrogenation, did not have to be even partly depressurized, if at all, in the course of the workup. This is because, after depressurization, the excess carbon dioxide-hydrogen mixture would have to be compressed again at substantial cost in terms of energy.

The problem accordingly was that of providing a preferably continuous, integrated process for production of formamide and its alkyl, cycloalkyl, aryl and aralkyl derivatives on the basis of the starting materials carbon dioxide, hydrogen and ammonia, mono- or diamines. The target products formamide and its derivatives should be made available at high yields and selectivities. The workup of the reaction effluent from the reactor should be technically simple, have low energy requirements and exclusively involve substances which are already part of the process, i.e., no additional auxiliary substances. Removal and return of the homogeneous hydrogenation catalysts into the synthesis stage should be very efficient.

### BRIEF SUMMARY OF THE INVENTION

This problem is solved by a process for production of formamides of formula Ia

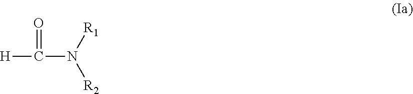

where R₁ and R₂ are each independently selected from the group consisting of hydrogen, linear or branched radicals having from 1 to 15 carbon atoms, cycloaliphatic radicals having from 5 to 10 carbon atoms, a substituted or unsubstituted phenyl radical and a phenylalkyl radical, and R₁ and R₂ may be combined to form a five- or six-membered ring comprising an oxygen atom, an N—H radical or an N—R₁ radical, where R₁ is as defined above,
by reacting carbon dioxide (1) with hydrogen (2) in a hydrogenation reactor I in the presence of
   a catalyst comprising an element from group 8, 9 or 10 of the periodic table,
   a tertiary amine of the formula NR₃R₄R₅ (IVa), where R₃ to R₅ are the same or different and each is independently selected from the group consisting of a branched or unbranched, cyclic or acyclic, aliphatic, aromatic or araliphatic radical having in each case from 1 to 16 carbon atoms and preferably from 1 to 12 carbon atoms, where individual carbon atoms may also be independently substituted by a hetero group selected from the group consisting of —O— and >N—, and also two or all three radicals may be linked together to form a chain comprising at least four atoms in each case, with the proviso that the total number of carbon atoms per molecule is at least 6, and also
   a polar solvent
to form formic acid-amine adducts as intermediates of formula IIa

NR₃R₄R₅.xᵢHCOOH (IIa),

(xᵢ=0.4 to 5)

wherein two liquid phases form in said hydrogenation reactor I in the course of the reaction, viz., a bottom phase comprising predominantly the polar solvent, in which the formic acid-amine adducts are enriched, and also a top phase comprising predominantly the tertiary amine, in which the homogeneous catalyst is enriched, and wherein
the effluent (3) from said hydrogenation reactor I is worked up in accordance with the following process steps:
   separating the two liquid phases from said hydrogenation reactor in a phase separation vessel,
   returning said top phase from said phase separation vessel into said hydrogenation reactor, and
   forwarding said bottom phase from said phase separation vessel into an extraction unit, in which:
residues of catalysts are extracted with the same tertiary amine as used in the hydrogenation and the tertiary amine laden with catalyst is recycled into said hydrogenation reactor I,
   which comprises
   (i) forwarding the catalyst-free stream comprising the formic acid-amine adducts of formula (IIa) and polar solvent (7) into a reactor IV, and therein reacting the formic acid-amine adducts of formula (IIa) at temperatures of 0 to 200° C. with ammonia or amines of formula (IIIa),

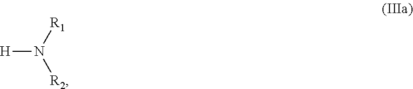

where R₁ and R₂ are each as defined under formula (Ia),
to obtain via the interstage having formula (Va) of the corresponding adduct of formic acid with ammonia or an amine of formula (IIIa)

NHR₁R₂.xHCOOH (Va)
the corresponding formamide of formula (Ia), and
(ii) separating the two-phase liquid reaction effluent from said reactor IV (stream 9) in a phase separation vessel V into a liquid phase (stream 10) enriched with formamides of formula (Ia) and a second liquid phase (stream 12) enriched with tertiary amine and wholly or partly returning the liquid phase (stream 12) enriched with tertiary amine into said extraction unit III, and
(iii) separating stream 10 in a distillation unit VI by distillation to recover said formamide of formula (Ia), remove water from the system, return the polar solvent (stream 8) into said hydrogenation reactor I and return the unconverted ammonia or amine of formula (IIIa) into said reactor IV (stream 11).

### BRIEF DESCRIPTION OF THE FIGURES

FIG. 1 shows a block diagram of a plant for a preferred process for production of formamides from carbon dioxide by the process of the present invention, and

FIG. 2 shows a block diagram for a more preferred process for production of formamides from carbon dioxide.

### DETAILED DESCRIPTION OF THE INVENTION

Preferably, the part stream (liquid phase enriched with tertiary amine) that is not returned into said extraction unit III is returned into said hydrogenation reactor I.

The catalyst to be used in the hydrogenation of carbon dioxide in the process of the present invention is preferably a homogeneous catalyst. This homogeneous catalyst comprises an element from the group 8, 9 or 10 of the periodic table, i.e., Fe, Co, Ni, Ru, Rh, Pd, Os, Ir and/or Pt. The catalyst preferably comprises Ru, Rh, Pd, Os, Ir and/or Pt, more preferably Ru, Rh and/or Pd and most preferably Ru.

The elements mentioned are present in the reaction mixture in the form of complexed compounds in homogeneous solution. The homogeneous catalyst must be selected such that it be present together with the tertiary amine in the liquid phase (B) in enriched form. “Enriched” here is to be understood as meaning a partition coefficient of the homogeneous catalyst.

P=[concentration of homogeneous catalyst in liquid phase (B)]/[concentration of homogeneous catalyst in liquid phase (A)] of >1. Here the liquid phase (A) is the phase comprising the solvent enriched with the formic acid-amine adducts. The homogeneous catalyst is generally selected via a simple test in which the partition coefficient of the desired homogeneous catalyst is experimentally determined under the process conditions planned.

Owing to its good solubility in tertiary amines, the process of the present invention preferably utilizes, as homogeneous catalysts, an organometallic complexed compound comprising an element from the group 8, 9 or 10 of the periodic table and at least one phosphine group having at least one branched or unbranched, cyclic or acyclic, aliphatic radical having from 1 to 12 carbon atoms, wherein individual carbon atoms may also be substituted by >P—. Branched cyclic aliphatic radicals accordingly also include radicals such as —CH₂—C₆H₁₁ for example. Examples of suitable radicals include methyl, ethyl, 1-propyl, 2-propyl, 1-butyl, 2-butyl, 1-(2-methyl)propyl, 2-(2-methyl)propyl, 1-pentyl, 1-hexyl, 1-heptyl, 1-octyl, 1-nonyl, 1-decyl, 1-undecyl, 1-dodecyl, cyclopentyl, cyclohexyl, cycloheptyl and cyclooctyl, methylcyclopentyl, methylcyclohexyl, 1-(2-methyl)pentyl, 1-(2-ethyl)hexyl, 1-(2-propyl)heptyl and norbornyl. Preferably, the branched or unbranched, cyclic or acyclic, aliphatic radical comprises at least 1 and preferably not more than 10 carbon atoms. In the case of an exclusively cyclic radical in the abovementioned sense, the number of carbon atoms is in the range from 3 to 12 and preferably at least 4 and also preferably not more than 8 carbon atoms. Ethyl, 1-butyl, sec-butyl, 1-octyl and cyclohexyl are preferred radicals.

The phosphine group may comprise one, two or three of the abovementioned branched or unbranched, cyclic or acyclic, aliphatic radicals. These can be the same or different. Preferably, the phosphine group comprises three of the abovementioned branched or unbranched, cyclic or acyclic, aliphatic radicals, and it is particularly preferable for the three radicals to all be the same. Preferred phosphines are P(n-CₙH₂ₙ₊₁)₃ where n is in the range from 1 to 10, and more preferably tri-n-butylphosphine, tri-n-octylphosphine and 1,2-bis(dicylohexylphosphino)ethane.

As mentioned, individual carbon atoms in the branched or unbranched cyclic or acyclic, aliphatic radicals mentioned can also be >P— substituted. Accordingly, multidentate, for example bi- or tridentate, phosphine ligands are co-comprised. These preferably comprise the >P—CH₂CH₂—P< moiety or the >P—CH₂CH₂— [symbol] —CH₂CH₂—P< moiety, respectively.

When the phosphine group further comprises radicals other than the above-mentioned branched or unbranched, cyclic or acyclic, aliphatic radicals, these other radicals generally correspond to those which are otherwise customarily used in phosphine ligands for organometallic complexed catalysts. Phenyl, tolyl and xylyl may be mentioned as examples.

The organometallic complexed compound may comprise one or more, for example two, three or four, of the abovementioned phosphine groups having at least one branched or unbranched, cyclic or acyclic, aliphatic radical. The remaining ligands of the organometallic complex can vary. Illustrative examples include hydride, fluoride, chloride, bromide, iodide, formate, acetate, propionate, carboxylate, acetylacetonate, carbonyl, DMSO, hydroxide, trialkylamine, alkoxide.

The homogeneous catalysts are obtainable in situ under reaction conditions not only directly in their active form but also by proceeding from customary standard complexes such as, for example, [M(p-cymene)Cl₂]₂, [M(benzene)Cl₂], [M(COD)(allyl)], [MCl₃xH₂O], [M(acetylacetonate)₃], [M(COD)Cl₂]₂, [M(DMSO)₄Cl₂] where M is an element from group 8, 9 or 10 of the periodic table by adding the corresponding phosphine ligand(s).

Homogeneous catalysts preferred for the process of the present invention are [Ru(Pn-Bu₃)₄(H)₂], [Ru(Pn-octyl₃)₄(H)₂], [Ru(Pn-Bu₃)₂(1,2-bis(dicyclohexylphosphino)-ethane)(H)₂], [Ru(Pn-octyl₃)₂(1,2-bis(dicyclohexylphosphino)ethane)(H)₂], [Ru(PEt₃)₄(H)₂. They provide turn over frequency (TOF) values of above 1000 h⁻¹ in the hydrogenation of carbon dioxide.

When homogeneous catalysts are used, the amount of the stated metal component being used in the organometallic complex is generally in the range from 0.1 to 5000 weight ppm, preferably in the range from 1 to 800 weight ppm and more preferably in the range from 5 to 800 weight ppm, all based on the entire liquid reaction mixture in the hydrogenation reactor.

The post-hydrogenation partition coefficient of the homogeneous catalyst based on the amount of ruthenium in the amine phase and the product phase comprising the formic acid-amine adduct is in the range of P above 0.5, preferably above 1.0 and more preferably above 4.

The tertiary amine is selected, and matched with the polar solvent, such that the hydrogenation effluent will form two liquid phases and the tertiary amine will be present in the top phase in the hydrogenation reactor in enriched form. “In enriched form” here is to be understood as meaning a weight fraction of >50% of the free tertiary amine, i.e., tertiary amine not bound in the form of the formic acid-amine adduct, in the top phase based on the total amount of free, tertiary amine in the two liquid phases. The weight fraction is preferably >90%. The tertiary amine is generally selected via a simple test in which the solubility of the desired tertiary amine in the two liquid phases is experimentally determined under the process conditions planned. The top phase may further comprise portions of the polar solvent and also of an apolar inert solvent.

The tertiary amine preferable for use in the process of the present invention is an amine of the general formula (IVa)

NR₃R₄R₅ (IVa),

where the R₃ to R₅ radicals are the same or different and are each independently a branched or unbranched, cyclic or acyclic, aliphatic, aromatic or araliphatic radical having in each case from 1 to 16 carbon atoms and preferably from 1 to 12 carbon atoms, wherein individual carbon atoms can independently of each other also be substituted by a hetero group selected from the group consisting of —O— and >N— and also two or all three radicals can also be linked together to form a chain comprising at least four atoms in each case, with the proviso that the total number of carbon atoms is at least 6.

Examples of suitable amines of formula (IVa) include:
triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, tri-n-undecylamine, tri-n-dodecylamine, tri-n-tridecylamine, tri-n-tetradecylamine, tri-n-pentadecylamine, tri-n-hexadecylamine, tri(2-ethylhexyl)amine;
dimethyldecylamine, dimethyldodecylamine, dimethyltetradecylamine, ethyldi(2-propyl)-amine, dioctylmethylamine, dihexylmethylamine;
tricyclopentylamine, tricyclohexylamine, tricycloheptylamine, tricyclooctylamine and their derivatives substituted by one or more methyl, ethyl, 1-propyl, 2-propyl, 1-butyl, 2-butyl- or 2-methyl-2-propyl groups;
dimethylcyclohexylamine, methyldicyclohexylamine, diethylcyclohexylamine, ethyl-dicyclohexylamine, dimethylcyclopentylamine, methyldicyclopentylamine;
triphenylamine, methyldiphenylamine, ethyldiphenylamine, propyldiphenylamine, butyldiphenylamine, 2-ethylhexyldiphenylamine, dimethylphenylamine, diethyl-phenylamine, dipropylphenylamine, dibutylphenylamine, bis(2-ethylhexyl)phenylamine, tribenzylamine, methyldibenzylamine, ethyldibenzylamine and their derivatives substituted by one or more methyl, ethyl, 1-propyl, 2-propyl, 1-butyl, 2-butyl or 2-methyl-2-propyl groups;
N—C₁— to —C₁₋₂-alkyl-piperidines, N,N-di-C₁- to C₁₋₂-alkylpiperazines, N—C₁— to —C₁₂-alkyl-pyrrolidines, N—C₁— to —C₁₋₂-alkylimidazoles and their derivatives substituted by one or more methyl, ethyl, 1-propyl, 2-propyl, 1-butyl, 2-butyl or 2-methyl-2-propyl groups;
1,8-diazabicyclo[5.4.0]undec-7-ene (“DBU”), 1,4-diazabicyclo[2.2.2]octane (“DABCO”), N-methyl-8-azabicyclo[3.2.1]octane (“tropane”), N-methyl-9-azabicyclo[3.3.1]nonane (“granatane”), 1-azabicyclo[2.2.2]octane (“quinuclidine”).

The process of the present invention can also utilize mixtures of any number of different tertiary amines IVa, as will be appreciated.

The tertiary amine used in the process of the present invention is particularly preferably an amine of the general formula (IVa) where the R¹ to R³ radicals are each independently selected from the group consisting of C₁- to C₁₂-alkyl, C₅- to C₈cycloalkyl, benzyl and phenyl.

It is particularly preferable for the tertiary amine used in the process of the present invention to be a saturated amine, i.e., comprising single bonds only, of the general formula (IVa).

It is very particularly preferable for the tertiary amine used in the process of the present invention to be an amine of the general formula (IVa) where the R₃ to R₅ radicals are each independently selected from the group consisting of C₅- to C₈₋alkyl, more particularly tri-n-pentylamine, tri-n-hexylamine, tri-n-heptylamine, tri-n-octylamine, dimethylcyclohexylamine, methyldicyclohexylamine, dioctylmethylamine and dimethyldecylamine.

The tertiary amine used is more particularly an amine of the general formula (IVa) where the R¹ to R³ radicals are each independently selected from C₅₋alkyl and C₆₋alkyl.

Preferably, the tertiary amine is in a liquid state in all process stages of the process of the present invention. However, this is not a mandatory requirement. It would also be sufficient for the tertiary amine to at least be dissolved in suitable solvents. Suitable solvents are in principle those solvents which are chemically inert in respect of the hydrogenation of carbon dioxide and also the thermal splitting of the adduct and in which the tertiary amine and, if a homogeneous catalyst is used, the homogeneous catalyst also are readily soluble, but conversely polar solvent and also the formic acid-amine adduct are only sparingly soluble. Suitable solvents therefore include in principle chemically inert, apolar solvents such as, for example, aliphatic, aromatic or araliphatic hydrocarbons, for example octane and higher alkanes, toluene, xylenes.

The polar solvent to be used in the hydrogenation of carbon dioxide in the process of the present invention must be selected, and/or matched to the tertiary amine, such that the polar solvent is present in the bottom phase in enriched form. “In enriched form” is to be understood as meaning a weight fraction of >50% of the polar solvent in the bottom phase based on the total amount of polar solvent in the two liquid phases. The weight fraction is preferably >70%. The polar solvent is generally selected via a simple test in which the solubility of the desired polar solvent in the two liquid phases is experimentally determined under the process conditions planned.

The polar solvent may comprise a pure polar solvent but also a mixture of various polar solvents, as long as the abovementioned solvent requirements in respect of boiling point and phase behavior are satisfied.

Classes of compounds useful as polar solvents are preferably alcohols and diols and also their formic esters and water.

Suitable alcohols dissolve the trialkylammonium formates preferentially in admixture with water to form a product phase having a miscibility gap to the free trialkylamine. Examples of suitable alcohols include methanol, ethanol, 2-methoxyethanol, 1-propanol, 2-propanol, 1-butanol, 2-butanol, 2-methyl-1-propanol. The ratio of alcohol to water must be chosen such that their combination with the trialkylammonium formate and the trialkylamine results in the formation of a two-phase mixture in which the bulk of the trialkylammonium formate, the water and also the polar solvent end up in the bottom phase, as is generally determined by a simple test in which the solubility of the desired polar solvent mixture in the two liquid phases is experimentally determined under the process conditions planned.

Classes of compounds useful as polar solvents preferably include diols and also their formic esters, polyols and also their formic esters, sulfones, sulfoxides, open-chain or cyclic amides and also mixtures thereof.

Examples of suitable diols and polyols include ethylene glycol, diethylene glycol, triethylene glycol, polyethylene glycol, 1,3-propanediol, 2-methyl-1,3-propanediol, 1,4-butanediol, dipropylene glycol, 1,5-pentanediol, 1,6-hexanediol and glycerol.

Tetramethylene sulfone (sulfolane) is mentioned as a suitable sulfone in particular.

Examples of suitable sulfoxides include dialkyl sulfoxides, preferably C₁- to C₆-dialkyl sulfoxides, more particularly dimethyl sulfoxide.

Examples of suitable open-chain or cyclic amides include formamide, N-methylformamide, N,N-dimethylformamide, N-methylpyrrolidone, acetamide and N-methylcaprolactam.

The molar ratio of the polar solvent or solvent mixture to be used in the process of the present invention to the tertiary amine used in the process of the present invention is generally in the range from 0.5 to 30 and preferably in the range from 1 to 20.

The carbon dioxide used in the hydrogenation of carbon dioxide can be used in solid, liquid or gaseous form. It is also possible to use gas mixtures comprising carbon dioxide which are available on a large industrial scale, provided they are substantially free of carbon monoxide (volume fraction <1% of CO). The hydrogen to be used in the hydrogenation of carbon dioxide is generally gaseous. Carbon dioxide and hydrogen may further comprise inert gases, such as nitrogen or noble gases for example. Advantageously, however, the level of these is below 10 mol % based on the total amount of carbon dioxide and hydrogen in the hydrogenation reactor. Larger amounts may optionally likewise still be tolerable, but generally necessitate the employment of a higher pressure in the reactor, necessitating further energy for compression.

The hydrogenation of carbon dioxide is preferably carried out in the liquid phase at a temperature in the range from 20 to 200° C. and an overall pressure in the range from 0.2 to 30 MPa abs. The temperature is preferably at least 30° C. and more preferably at least 40° C. and also preferably at most 150° C., more preferably at most 120° C. and even more preferably at most 80° C. The overall pressure is preferably at least 1 MPa abs and more preferably at least 5 MPa abs and also preferably at most 15 MPa abs.

The carbon dioxide partial pressure is generally at least 0.5 MPa and preferably at least 2 MPa and also generally at most 8 MPa. The hydrogen partial pressure is generally at least 0.5 MPa and preferably at least 1 MPa and also generally at most 25 MPa and preferably at most 10 MPa.

The molar ratio of hydrogen to carbon dioxide in the feed to the hydrogenation reactor is preferably in the range from 0.1 to 10 and more preferably in the range from 1 to 3.

The molar ratio of carbon dioxide to tertiary amine in the feed to the hydrogenation reactor is generally in the range from 0.1 to 10 and preferably in the range from 0.5 to 3.

The hydrogenation reactor used can in principle be any reactor which in principle is useful for gas/liquid reactions under the stated temperature and the stated pressure. Suitable standard reactors for liquid-liquid reaction systems are indicated for example in K. D. Henkel, “Reactor Types and Their Industrial Applications”, in Ullmann's Encyclopedia of Industrial Chemistry, 2005, Wiley-VCH Verlag GmbH & Co. KGaA, DOI: 10.1002/14356007.b04_{—}087, chapter 3.3 “Reactors for gas-liquid reactions”. Stirred tank reactors, tube reactors and bubble column reactors may be mentioned as examples.

The hydrogenation of carbon dioxide in the process of the present invention can be carried out as a batch operation or as a continuous operation. For batch operation, the reactor is charged with the desired liquid and optionally solid starting and auxiliary materials before carbon dioxide and hydrogen are injected to the desired pressure at the desired temperature. After the reaction has ended, the reactor is generally depressurized and the two liquid phases which have formed are separated from each other. For continuous operation, the starting and auxiliary materials including the carbon dioxide and hydrogen are added continuously. Correspondingly, the liquid phase is discharged from the reactor continuously, so that the level of liquid in the reactor remains on average the same. Continuous hydrogenation of carbon dioxide is preferred.

The average residence time in the hydrogenation reactor is generally in the range from 10 minutes to 5 hours.

The formic acid-amine adducts formed in the course of the hydrogenation of carbon dioxide in the presence of the catalyst to be used, of the polar solvent and of the tertiary amine generally have the general formula (IIa)

NR₃R₄R₅.xᵢHCOOH (IIa),

where the R₃ to R₅ radicals correspond to the radicals described for the tertiary amine (IVa) and xᵢ is in the range from 0.4 to 5 and preferably in the range from 0.7 to 1.6. The particular averaged compositions of the amine-formic acid ratios in the product phases of the particular process steps, i.e., the factor xᵢ, can be for example determined by determining the formic acid content by titration with an alcoholic KOH solution against phenolphthalein and the amine content via gas chromatography. The composition of the formic acid-amine adducts, i.e., the factor xᵢ, can change during the various process steps. For instance, adducts having a higher formic acid content where x₂>x₁ and x₂ is in the range from 1 to 4 generally develop after removal of the polar solvent, while the excess, free amine can form a secondary phase.

The hydrogenation of carbon dioxide by the process of the present invention leads to the formation of two liquid phases. The bottom phase is enriched with the formic acid-amine adducts and also the polar solvent. With regard to the formic acid-amine adducts, “enriched” is to be understood as meaning a partition coefficient for the formic acid-amine adducts

P=[concentration of formic acid-amine adduct (II) in liquid phase (A)]/[concentration of formic acid-amine adduct (II) in liquid phase (B)]

of >1, while A and B are each as defined above. The partition coefficient is preferably ≧2 and more preferably ≧5. The top phase is enriched with the tertiary amine. When a homogeneous catalyst is used, it will likewise be present in the top phase in enriched form.

The two liquid phases formed are separated in the course of the process of the present invention and the top phase is returned to the hydrogenation reactor. It is also advantageous to return to the hydrogenation reactor any further liquid phase present above the two liquid phases and comprising unconverted carbon dioxide and also any gas phase comprising unconverted carbon dioxide and/or unconverted hydrogen. It is optionally desirable, for example to remove undesired by-products or impurities from the process, for part of the top phase and/or part of the liquid or gaseous phases comprising carbon dioxide or carbon dioxide and hydrogen to be removed from the process.

The two liquid phases are generally separated via gravimetric phase separation. Useful phase separation vessels include for example standard apparatuses and standard methods as found for example in E. Müller et al., “Liquid-Liquid Extraction”, in Ullmann's Encyclopedia of Industrial Chemistry, 2005, Wiley-VCH Verlag GmbH & Co. KGaA, DOI:10.1002/14356007.b03_{—}06, chapter 3 “Apparatus”. Generally, the liquid phase enriched with the formic acid-amine adducts and also the polar solvent is heavier and forms the bottom phase.

Phase separation can take place for example after depressurization, for example to about or close to atmospheric pressure, and cooling of the liquid reaction mixture, for example to about or close to ambient temperature. However, there is a risk that at least part of the gas, particularly carbon dioxide, dissolved in the liquid phases at the higher reaction pressure will degas in the course of depressurization and will have to be separately compressed, and returned to the hydrogenation reactor, as a gas stream. Similarly, the bottom phase also has to be brought separately to reaction pressure before being returned to the hydrogenation reactor. And the gas and liquid phases to be returned each require a suitable compressor designed according to the pressure difference to be overcome, or respectively a pump which moreover consumes additional energy in operation.

The present inventors have found that the two liquid phases in the present system, i.e., a bottom phase enriched with the formic acid-amine adducts and also the polar solvent and a top phase enriched with the tertiary amine and also, if used, a homogeneous catalyst, are very readily separated from each other even at a distinctly elevated pressure, given a suitable combination of solvent and amine. Therefore, solvent and amine are preferably selected in the process of the present invention such that the separation of the bottom phase, enriched with the formic acid-amine adducts and the polar solvent, from the other, top phase enriched with the tertiary amine, and also the returning of the top phase to the hydrogenation reactor can be carried out at a pressure in the range from 1 to 30 MPa abs. In terms of the overall pressure in the hydrogenation reactor, the pressure is preferably at most 15 MPa abs. It may even be possible to separate the two liquid phases from each other without prior depressurization and to return the top phase to the hydrogenation reactor without significant pressure elevation. In this case and also in the case of a but minimal depressurization, it is possible to dispense entirely with returning any gas phase. Whether this omission is possible for a particular given system can be determined in advance, if in doubt, via simple experimental examples.

The process of the present invention can thus preferably be carried out in such a way that the pressure in the hydrogenation reactor and in the phase separation vessel is the same or substantially the same.

The process of the present invention can thus preferably be carried out in such a way that the pressure and the temperature in the hydrogenation reactor and in the phase separation vessel are the same or substantially the same, substantially the same meaning a pressure difference of up to +/−0.5 MPa and a temperature difference of up to +/−5° C.

Phase separation is particularly preferably performed at a pressure of at least 50%, even more particularly preferably of at least 90% and especially of at least 95% of the reaction pressure. The pressure at phase separation is more preferably equal to at most 105% and even more preferably equal to at most 100% of the reaction pressure.

It was also found that, surprisingly, the two liquid phases in the present system can also be separated from each other very readily even at an elevated temperature equal to the reaction temperature. Hence phase separation does not require any cooling and subsequent heating of the top phase to be returned, which likewise saves energy.

The findings concerning phase separation under elevated pressure and elevated temperature are if anything surpassed by the finding that specifically the top phase of the system according to the present invention can have, under superatmospheric pressure, a particularly high absorption capacity for carbon dioxide provided the amine and the polar solvent are suitable choices. In other words, any excess carbon dioxide, unconverted in the hydrogenation reaction, will be highly preferentially present in the top phase and hence readily returnable as a liquid into the reactor.

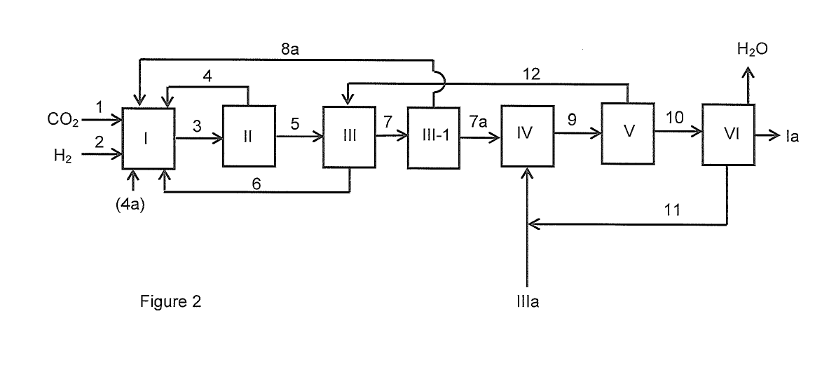

The bulk of the polar solvent of the removed bottom phase is thermally separated from the formic acid-amine adducts in a distillation unit, and the distillatively removed polar solvent is returned to the hydrogenation reactor. The pure formic acid-amine adducts and also free amine are obtained in the pot of the distillation unit, since when the polar solvent is removed formic acid-amine adducts having a relatively low amine content are formed, as a result of which a two-phase bottoms mixture consisting of an amine phase and a formic acid-amine adduct phase is formed (FIG. 2).

The thermal removal of the polar solvent or solvent mixture, see above, is preferably carried out at a pot temperature at which, at the given pressure, no free formic acid is formed from the formic acid-amine adduct having the higher (×1) or lower (×2) amine content. In general, the pot temperature of the thermal separating unit is at least 20° C., preferably at least 50° C. and more preferably at least 70° C. and also generally at most 210° C. and preferably at most 190° C. The pressure is generally at least 0.0001 MPa abs, preferably at least 0.005 MPa abs and more preferably at least 0.01 MPa abs and also generally at most 1 MPa abs and preferably 0.1 MPa abs.

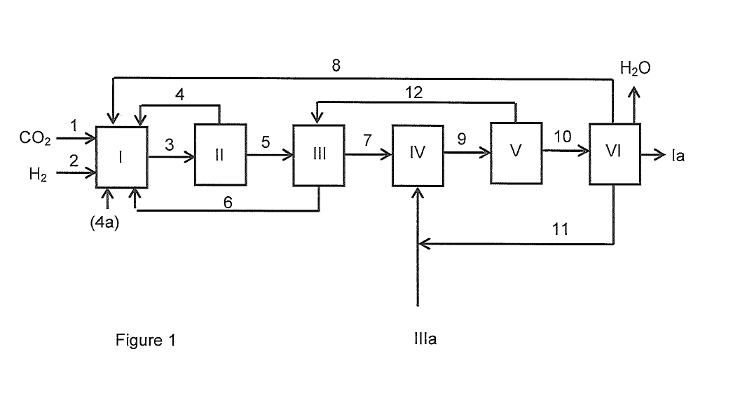

The thermal removal of the polar solvent or solvent mixture is effected either in an evaporator or in a distillation unit consisting of a vaporizer and a column packed with ordered packing, random packing and/or trays. The solvent can be condensed after the thermal separation, in which case the enthalpy released in the course of the condensation can in turn be used to, for example, preheat the ex-extraction solvent with amine-formic acid adduct mixture to evaporation temperature (FIG. 1).

Alternatively, only parts of the solvent mixture can be separated off. The polar solvent can also be separated off only in sub-step VI and returned into hydrogenation stage I.

The solution of the adduct of tertiary amine and formic acid is extracted with streams of free tertiary amine which come from the appropriate phase separation vessels, and returned into the hydrogenation reactor. This is done in order that residual amounts of hydrogenation catalyst may be removed from the product stream. This extraction provides efficient recovery of the costly, active noble metal catalyst for the hydrogenation reaction.

The extraction is carried out at temperatures in the range from 30 to 100° C. and pressure in the range from 0.1 to 8 MPa. The extraction can also be carried out under hydrogen pressure.

The extraction of the hydrogenation catalyst can be carried out in any suitable apparatus known to a person skilled in the art, preferably in countercurrent extraction columns, mixer-settler cascades or combinations of mixer-settlers.

Fractions of individual components of the polar solvent from the liquid phase to be extracted may become dissolved in the extractant, the amine stream, in addition to the catalyst. This is not a disadvantage for the process since the already extracted amount of solvent does not have to be fed to the solvent removal stage and thus may save evaporation energy.

An apparatus for adsorbing traces of hydrogenation catalyst may advantageously be integrated between the extraction apparatus and the thermal separating apparatus. Numerous adsorbents are suitable for the adsorption. Examples are polyacrylic acid and salts thereof, sulfonated polystyrenes and salts thereof, activated carbons, montmorillonites, bentonites, silica gels and also zeolites.

When the amount of hydrogenation catalyst in the product stream from phase separation vessel II is less than 1 ppm and more particularly less than 0.1 ppm, the adsorption apparatus will be sufficient to remove and recover the hydrogenation catalyst. The extraction stage can then be omitted and the tertiary amine can be returned into the hydrogenation stage together with the organic solvent.

Subsequently, the formic acid adducts of formula (IIa) are reacted with ammonia (R₁ and R₂ in formula (IIIa)=hydrogen) or amines of formula (IIIa) to form formamides of formula (Ia), tertiary amines IVa and water:

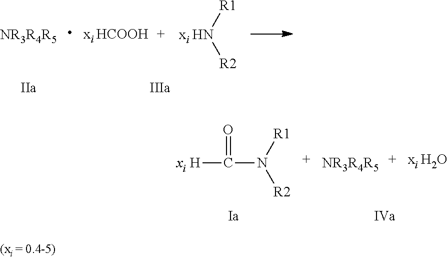

In a preferred embodiment of the present invention, the reaction is carried out in the presence of the polar solvent used in the hydrogenation reactor. Preferably, the entire quantity of polar solvent arriving in the reactor is used.

The reaction is preferably carried out at temperatures in the range from 80 to 200° C., preferably in the range from 100 to 180° C., and more preferably in the range from 120 to 170° C. The pressure is in the range from 0.01 to 10 MPa, preferably in the range from 0.1 to 7 MPa and more preferably in the range from 0.2 to 5 MPa.

The reaction can also be carried out at temperatures in the range from 0 to 80° C. in the pressure ranges specified. The reaction effluents formed comprise, in addition to formamides of formula Ia, quite overwhelmingly as an intermediate stage, formates of formula Va, i.e., adducts of formic acid with ammonia or an amine of formula (IIIa).

NHR₁R₂.xHCOOH (Va).

These mixtures can be converted into formamides of formula (Ia) at temperatures between 80 and 200° C. like the adducts (IIa).

The molar ratio of formic acid in adduct IIa to ammonia or amine IIIa is 1:5, preferably 1:2 and more preferably 1:1.

The reaction can be carried out batchwise, but is preferably carried out continuously.

The synthesis of the formamides Ia can be carried out in standard reactors, as specified for example in K. D. Henkel, “Reactor Types and their Industrial Applications” in Ullmanns Encyclopedia of Industrial Chemistry, 2005, Wiley-VCH Verlag, chapter 3.3. Stirred tank reactors, tube reactors and bubble column reactors are mentioned as examples.

In a further preferred embodiment of the present invention, the whole or part of the polar solvent is distillatively removed before feeding to the formamide reactor and returned into the hydrogenation reactor (I). The removed amount of polar solvent can be in the range from 25 to 99%, preferably in the range from 35 to 97% and more preferably in the range from 50 to 95% of the polar solvent quantity present in the stream prior to feeding to the reactor.

This version of the process is for example advantageous when primary alcohols such as methanol are used together with water as polar solvents in the hyrogenation of carbon dioxide,

It is also possible in principle, and can be advantageous, to use a polar solvent in the hydrogenation step in hydrogenation reactor I other than in the amidation step, in reactor IV. To this end, the polar solvent is separated from the hydrogenation and recycled into hydrogenation reactor I. The amidation stage utilizes a polar solvent in reactor IV other than that used in the hydrogenation step, and it is separated off again after the amidation has taken place, and is recycled into reactor IV, where the amidation is carried out. For instance, the carbon dioxide hydrogenation can be carried out in the presence of methanol and water and the reaction of adduct (IIa) with ammonia can be carried out in the presence of sulfolane. Polar solvents particularly suitable for the amidation include for example sulfones, sulfoxides or open-chain or cyclic amides.

The distillation can take place at atmospheric pressure or under reduced pressure when low-boiling polar solvents such as the monohydric alcohols methanol, ethanol, propanols and butanols are to be separated off. By contrast, operation under reduced pressure is preferable when diols are used as polar solvents.

Different apparatuses can be used as distillation units, depending on the separation problem. When the boiling points of adduct IIa and of the polar solvent are far apart, evaporators such as falling film evaporators can be used for example. However, the distillation unit to be used generally comprises a distillation column containing random packing, ordered packing and/or trays.

The reaction of formic acid adducts of formula (IIa) with ammonia or amines of formula (IIIa) according to the process of the present invention leads both in the presence and in the absence of polar solvents to the formation of two liquid phases C and D. Liquid phase C is enriched with the formamide of formula (Ia), water and any unconverted ammonia or amine IIIa. With regard to the formamide of formula (Ia), enriched is to be understood as meaning a partition coefficient

P=[concentration of formamide of formula (Ia) in liquid phase C]/[concentration of formamide of formula (Ia) in liquid phase D]

of >1. The partition coefficient is preferably ≧2 and more preferably ≧5. The liquid phase D is enriched with the tertiary amine emerging out of the adduct IIa.

The two liquid phases C and D formed are separated from each other in the process of the present invention. The liquid phase D is returned into the extraction apparatus and is used there to extract residual quantities of homogeneous, dissolved hydrogenation catalyst. It is optionally desirable to remove part of the liquid phases C or D from the process if unwanted by-products or impurities are to be removed from the process for example.

The two liquid phases C and D are generally separated via gravimetric phase separation. Suitable for this are for example standard apparatuses and standard methods as can be found for example in E. Müller et. al., “Liquid-Liquid Extraction”, in Ullmann's Encyclopedia of Industrial Chemistry, 2005, Wiley-VCH Verlag GmbH & Co. KGaA, DOI: 10.1002/14356007.b03_{—}06, chapter 3 “Apparatus”.

Phase separation can take place for example after depressurization, for example to about or close to atmospheric pressure, and cooling of the liquid reaction mixture, for example to about or close to ambient temperature.

The liquid phase C is directed into a distillation unit VI and worked up there by distillation. It comprises the target product formamide of formula (Ia), any unconverted ammonia or amine of formula (IIIa), water of reaction and polar solvents selected from the group consisting of methanol, ethanol, propanols and butanols or from the group of diols. In one version of this process, the polar solvents are separated off upstream of the feed to the formamide reactor and returned into hydrogenation stage I.

Distillation unit VI consists of at least one and preferably two to three distillation columns. These columns contain for example random packing, ordered packing and/or trays, depending on the separation problems.

When the polar solvents were not separated off beforehand, there needs to be awareness in the case of ethanol of the ethanol-water and propanol-water homoazeotropes and in the case of butanols of heteroazeotropes. The water of reaction is removed from the process. Polar solvents are returned into the hydrogenation stage and unconverted ammonia and unconverted amines IIIa are returned into the reactor. The target products, the formamides of formula (Ia), are removed from the process and, if necessary, sent to a preferably distillative final purification stage.

In a further preferred embodiment, adducts of formula (IIa), formates of formula (Va) or mixtures of adducts of formula (IIa) and formates of formula (Va) are reacted with amines of formula (IIIa), preferably with ammonia and polar solvents under distillation conditions. The reaction takes place in a distillation apparatus, for example a distillation flask with emplaced distillation column. Useful apolar solvents include aliphatic, cycloaliphatic or aromatic hydrocarbons, such as n-heptane, n-hexane, cyclohexane, methylcyclohexane, toluene, ethylbenzene, ortho-, meta- and para-xylene and mixtures thereof. These compounds form heteroazeotropes with water. The pot temperatures of this distillation range from 80 to 200° C., preferably from 110 to 200° C. and more preferably from 135 to 190° C. The pressure is in the range from 0.01 MPa to 10 MPa, preferably in the range from 0.1 MPa to 7 MPa and more preferably in the range from 0.2 to 5 MPa. The water of reaction is distilled off together with the polar solvent. The liquid two-phase mixture formed in the course of the condensation has water removed from it, which is removed from the system, while the apolar solvent is returned into the distillation. The bottom product of the distillation is worked up. Formamide of formula (Ia) is separated from tertiary amine of formula IVa. The tertiary amine IVa is returned into hydrogenation stage I.

The process of the present invention has particular advantages when practiced on a large scale industrial manufacturing plant built with “economy of scale” for production of adducts of formula (IIa) from tertiary amines and formic acid to produce formic acid and formamides Ia.

The invention will now be more particularly elucidated with reference to exemplary embodiments and a drawing.

### Exemplary Embodiments

### 1. Carbon Dioxide Hydrogenation (in Hydrogenation Reactor I) and Phase Separation of Hydrogenation Reactor Effluent (in Phase Separation Vessel II) (Examples A1 to A4)

A 250 ml autoclave made of Hastelloy C and equipped with a magnetic stirbar was charged under inert conditions with the tertiary amine, polar solvent and homogeneous catalyst specified in each case in the following table 1. The autoclave was subsequently sealed and CO₂ was injected at room temperature. This was followed by injection of H₂ and heating of the reactor under agitation (700 rpm). After the appropriate reaction time, the autoclave was cooled down and the reaction mixture was depressurized to obtain a two-phase product mixture wherein the top phase was enriched with free tertiary amine and the homogeneous catalyst and the bottom phase was enriched with the polar solvent and the formic acid-amine adduct formed. The total formic acid content in the formic acid-amine adduct was determined by potentiometric titration with 0.1 N KOH in methanol using a Mettler Toledo DL50® titrator. The result was used to compute the turn over frequency (TOF) and the reaction rate. The composition of the two phases was determined via gas chromatography. The ruthenium content was determined by atomic absorption spectroscopy (AAS). The parameters and the results of the individual runs are shown in table 1.

The two liquid phases were separated from each other in a separation funnel.

Examples A-1 to A-4 show that high to very high reaction rates of up to 0.98 mol kg⁻¹ h⁻¹ (reaction rate=mol of formic acid/(kg of weight of reaction effluent×h reaction time) are obtained. The systems investigated all formed two phases, the top phase being in each case enriched with the free tertiary amine and the homogeneous catalyst and the bottom phase in each case being enriched with the polar solvent and the formic acid-amine adduct formed.

TABLE 1

| | Example A-1 | Example A-2 | Example A-3 |
|---|---|---|---|
| Tertiary amine | 75.0 g of trihexylamine | 75.0 g of trihexylamine | 75.0 g of trihexylamine |
| Polar solvent (used) | 24.0 g of methanol | 25.0 g of ethanol | 25.0 g of 1-propanol |
| | 6.7 g of water | 6.0 g of water | 6.0 g of water |
| Catalyst | 0.18 g of [Ru(Pn-Bu₃)₄(H)₂] | 0.16 g of [Ru(Pn-Oct₃)₄(H)₂], | 0.16 g of [Ru(Pn-Oct₃)₄(H)₂], |
| | | 0.08 g of 1,2-bis(dicyclohexyl- | 0.08 g of 1,2-bis(dicyclohexyl- |
| | | phosphino)ethane | phosphino)ethane |
| Injection of CO2 | with 20.2 g to 3.5 MPa abs | with 19.9 g to 2.5 MPa abs | with 19.9 g to 2.5 MPa abs |
| Injection of H2 | to 11.5 MPa abs | to 11.5 MPa abs | to 10.5 MPa abs |
| Heating | to 50° C. | to 50° C. | to 50° C. |
| Pressure change | to 11.0 MPa abs | to 10.5 MPa abs | to 11.3 MPa abs |
| Reaction time | 1 hour | 1 hour | 1 hour |
| Top phase | 44.1 g | 60.2 g | 46.4 g |
| | 2.7% of methanol | 0.7% of water | 2.0% of water |
| | 97.3% of trihexylamine | 6.6% of ethanol | 6.2% of 1-propanol |
| | | 92.7% of trihexylamine | 91.8% of trihexylamine |
| Bottom phase | 65.9 g | 51.3 g | 60.6 g |
| | 7.5% of formic acid | 5.3% of formic acid | 5.6% of formic acid |
| | 10.2% of water | 9.3% of water | 8.4% of water |
| | 34.6% of methanol | 41.0% of ethanol | 36.5% of 1-propanol |
| | 47.7% of trihexylamine | 44.4% of trihexylamine | 49.5% of trihexylamine |
| KRu (cRu in top phase/ | 1.9 | 1.6 | 1.5 |
| cRu in bottom phase) | | | |
| TOF | 551 h⁻¹ | 569 h⁻¹ | 726 h⁻¹ |
| Reaction rate | 0.98 mol kg⁻¹ h⁻¹ | 0.53 mol kg⁻¹ h⁻¹ | 0.68 mol kg⁻¹ h⁻¹ |

### 2. Extraction of Residual Catalyst Quantities (in Extraction Apparatus III) (Example B-1)

Example B-1, table 2 shows that the phase separation of a hydrogenation effluent gives 26.2 g of a bottom phase comprising 6.1% by weight of formic acid in the form of the formic acid-amine adduct and 33 ppm of ruthenium. This bottom phase was stirred three times for 10 minutes with 26.2 g of tri-n-hexylamine each time at room temperature under inert conditions. The phases were separated. The ruthenium content of the extracted bottom phase, as determined by AAS analysis, was 21 ppm of ruthenium.

Example B-1 shows that the amount of ruthenium in the bottom phase can be reduced by around 36% by extraction with the same tertiary amine as used in the CO₂ hydrogenation. The ruthenium content could be further reduced by additional extraction steps or a continuous countercurrent extraction.

TABLE 2

| Catalyst extraction | Example B-1 |
|---|---|
| Tertiary amine | 37.5 g of trihexylamine |
| Polar solvent (used) | 12.0 g of methanol |
| | 0.5 g of water |
| Catalyst | 0.16 g of [Ru(Pn-Octyl₃)₄(H)₂] |
| Injection of CO₂ | to 1.7 MPa |
| Injection of H₂ | to 8.0 MPa |
| Heating | 50° C. |
| Reaction time | 1.5 hours |
| Top phase | 23.3 g |
| Bottom phase | 26.2 g |
| | 6.1% of formic acid |
| c_{Ru} top phase after reaction | 350 ppm |
| c_{Ru} bottom phase after reaction | 33 ppm |
| c_{Ru} bottom phase after extraction | 21 ppm |

3. Distillative removal of polar solvent upstream of amide synthesis (in distillation apparatus III-1) (example C-1)

Example C-1 shows the distillative removal of the polar solvents methanol and water from a hydrogenation effluent. A rotary evaporator operating at 120° C. and 0.02 MPa yielded a distillate comprising the entire methanol, 0.3% by weight of formic acid and almost all of the water. The two-phase bottoms effluent (top phase+bottom phase) can be used for amide production in reactor IV (FIGS. 1 and 2).

TABLE 3

| Removal of polar solvent | Example C-1 |
|---|---|
| Feed mixture (% by weight) | 75 g of trihexylamine |
| Polar solvent (used) | 199.8 g: |
| | 8.9% of formic acid |
| | 28.4% of methanol |
| | 5.6% of water |
| | 57.1% trihexylamine |
| Formic acid: amine | 1:1.1 |
| Feed mixture | |
| Pressure | 0.02 MPa |
| Temperature | 120° C. |
| Bottom phase in pot after | 79.8 g: |
| distillation (% by weight) | 22.1% of formic acid |
| | 1.5% of water |
| | 76.4% of trihexylamine |
| Top phase in pot after distillation | 50.5 g: |
| (% by weight) | 100% of trihexylamine |
| Distillate | 66.6 g: |
| | 0.3% of formic acid |
| | 81.2% of methanol |
| | 18.5% of water |

4. Production of formamides by reaction of adducts of formic acid with tertiary amines with primary and secondary amine (n-butylamine or dimethylamine) or with ammonia (in reactor IV)

A 250 ml autoclave made of Hastelloy C and fitted with a magnetic stirbar was charged under inert conditions with the formic acid-tert-amine adduct IIa and optionally a polar solvent and water (see table 4, examples D-1 to D-19). The autoclave was subsequently sealed. Dimethylamine, n-butylamine or ammonia were injected at room temperature. Thereafter, the reactor was heated under agitation (700 rpm). After the desired reaction time the autoclave was cooled down and the reaction mixture was depressurized to obtain a two-phase product mixture wherein the top phase consisted of the tertiary amine and the bottom phase consisted of the corresponding amide, water and optionally polar solvent and also optionally ammonium formate. The two phases were separated with the aid of a separation funnel and weighed. The composition of the two phases was determined via gas chromatography and also proton NMR spectroscopy. The parameters and results of the individual runs are shown in tables 4 to 6.

### 4.1 Production of N,N-Dimethylformamide (Examples D1 to D19)

TABLE 4

| | Example D-1 | Example D-2 | Example D-3 | Example D-4 | Example D-5 |
|---|---|---|---|---|---|
| Amine | 48 g of | 60 g of | 48 g of | 48 g of | 23 g of N-ethyl- |
| | trihexylamine | trihexylamine | trihexylamine | trihexylamine | diisopropylamine |
| Formic acid | 16 g of | 20 g of | 16 g of | 16 g of | 17 g of HCOOH |
| | HCOOH | HCOOH | HCOOH | HCOOH | |
| Solvent (LM) | 72 g of | no LM | 72 g of | no LM | 73 g of |
| | 1,4-butanediol | | 1,4-butanediol | | 1,4-butanediol |
| | | | 6 g of water | | |
| Dimethylamine | 18 g of DMA | 21 g of DMA | 19 g of DMA | 19 g of DMA | 16 g of DMA |
| (DMA) | | | | | |
| Pressure after | 0.33 MPa | 0.21 MPa | 0.36 MPa | 0.32 MPa | 0.28 MPa |
| injection | | | | | |
| Temperature | 150° C. | 150° C. | 150° C. | 150° C. | 130° C. |
| Run time | 4 hours | 2 hours | 2 hours | 2 hours | 2 hours |
| Top phase | 48 g of | 57 g of | 46 g of | 46 g of | 15 g of N-ethyl- |
| | trihexylamine | trihexylamine | trihexylamine | trihexylamine | diisopropylamine |
| Bottom phase | 102 g | 40 g | 112 g | 33 g | 109 g |
| Yield GC (area %) | 99.5% | 94.0% | 75.9% | 99.4% | 95.0% |
| dimethylformamide | | | | | |
| | Example D-6 | Example D-7 | Example D-8 | Example D-9 | Example D-10 |
| Amine | 48 g of | 48 g of | 48 g of | 25 g of | 48 g of |
| | trihexylamine | trihexylamine | trihexylamine | trihexylamine | trihexylamine |
| Formic acid | 16 g of HCOOH | 16 g of HCOOH | 16 g of HCOOH | 4 g of HCOOH | 16 g of HCOOH |
| Solvent | 72 g of MeOH | 72 g of MeOH | none | 25 g of methanol | 72 g of |
| | 6 g of water | | | 6 g of water | 1,5-pentanediol |
| Dimethylamine | 18 g of DMA | 19 g of DMA | 20 g of DMA | 4 g of DMA | 16 g of DMA |
| (DMA) | | | | | |
| Pressure after | 0.26 MPa | 0.09 MPa | 0.26 MPa | 0.29 MPa | 0.29 MPa |
| injection | | | | | |
| Temperature | 130° C. | 130° C. | 130° C. | 130° C. | 130° C. |
| Run time | 2 hours | 2 hours | 2 hours | 2 hours | 2 hours |
| Top phase | 48 g of | 46 g of | 46 g of | 23 g of | 47 g of |
| | trihexylamine | trihexylamine | trihexylamine | trihexylamine | trihexylamine |
| Bottom phase | 111 g | 107 g | 33 g | 25 g | 101 g |
| Yield GC (area %) | 60.8% | 82.9% | 99.4% | 20.7% | 89.5% |
| dimethylformamide | | | | | |
| | Example D-11 | Example D-12 | Example D-13 | Example D-14 | Example D-15 |
| Amine | 48 g of | 27 g of | 33 g of | 41 g of | 33 g of N,N- |
| | trihexylamine | tripropylamine | tributylamine | tripentylamine | dimethyl- |
| | | | | | decylamine |
| Formic acid | 16 g of | 17 g of HCOOH | 17 g of HCOOH | 17 g of HCOOH | 16 g of HCOOH |
| | HCOOH | | | | |
| Solvent | 73 g of | 72 g of | 72 g of | 72 g of | 72 g of |
| | 1,6-hexanediol | 1,4-butanediol | 1,4-butanediol | 1,4-butanediol | 1,4-butanediol |
| Dimethylamine | 17 g of DMA | 15 g of DMA | 16 g of DMA | 16 g of DMA | 16 g of DMA |
| (DMA) | | | | | |
| Pressure after | 0.32 MPa | 0.19 MPa | 0.18 MPa | 0.18 MPa | 0.21 MPa |
| injection | | | | | |
| Temperature | 130° C. | 130° C. | 130° C. | 130° C. | 130° C. |
| Run time | 2 hours | 2 hours | 2 hours | 2 hours | 2 hours |
| Top phase | 47 g of | 20 g of | 31 g of | 38 g of | 29 g of |
| | trihexylamine | tripropylamine | tributylamine | tripentylamine | N,N-dimethyl- |
| | | | | | decylamine |
| Bottom phase | 102 g | 106 g | 101 g | 101 g | 104 g |
| Yield GC (area %) | 83.0% | 87.2% | 87.3% | 86.2% | 88.3% |
| dimethylformamide | | | | | |

| | Example D-16 | Example D-17 | Example D-18 | Example D-19 |
|---|---|---|---|---|
| Amine | 35 g of N-methyl- | 23 g of N-ethyl- | 64 g of tris(2-ethylhexyl)- | 70 g of |
| | dicyclohexylamine | diisopropylamine | amine | trihexylamine |
| Formic acid | 17 g of HCOOH | 17 g of HCOOH | 16 g of HCOOH | 5 g of HCOOH |
| Solvent | 72 g of | 73 g of | 72 g of | 30 g of |
| | 1,4-butanediol | 1,4-butanediol | 1,4-butanediol | 1,4-butanediol |
| Dimethylamine (DMA) | 16 g of DMA | 16 g of DMA | 16 g of DMA | 5 g of DMA |
| Pressure after injection | 0.18 MPa | 0.28 MPa | 0.32 MPa | 0.30 MPa |
| Temperature | 130° C. | 130° C. | 130° C. | 130° C. |
| Run time | 2 hours | 2 hours | 2 hours | 2 hours |
| Top phase | 25 g of N-methyl- | 15 g of N-ethyl- | 64 g of tris(2-ethylhexyl)- | 63 g of |
| | dicyclohexylamine | diisopropylamine | amine | trihexylamine |
| Bottom phase | 112 g | 109 g | 101 g | 33 g |
| Yield GC (area %) | 86.4% | 95.0% | 89.7% | 17.9% |
| dimethylformamide | | | | |

The runs shown in table 4, for producing dimethylformamide from adducts IIa and dimethylamine (DMA) in the presence of 1,4-butanediol as polar solvent (D-1, D-15) and in the absence of a polar solvent and water (D-4, D-8), gave dimethylformamide yields of above 95%.

In the presence of primary alcohols such as methanol as polar solvent, the dimethylformamide yield decreased to 82.9% (D-7). The dimethylformamide yield went down even further in the presence of methanol and water. Runs D-6 and D-9 gave dimethylformamide yields of just 60.8% and 20.7%, respectively.

It is accordingly preferable for water, particularly preferably water together with primary alcohols as polar solvents, to be distillatively removed in process step III-1 and returned as stream 8 into the hydrogenation step.

### 4.2 Production of N-Butylformamide (Example E1)

TABLE 5

| | Example E-1 |
|---|---|
| Amine | 48 g of trihexylamine |
| Formic acid | 16 g of HCOOH |
| Solvent | 72 g of 1,4-butanediol |
| N-Butylamine | 31 g n-BuNH₂ |
| Pressure after injection | 0.12 of MPa |
| Temperature | 150° C. |
| Run time | 4 hours |
| Top phase | 35 g of trihexylamine |
| Bottom phase | 125 g |
| Yield GC (area %) | 99.5% of |
| butylformamide | butylformamide |

The experimental result of E-1 shows that the reaction of adduct IIa with n-butylamine in the presence of 1,4-butanediol as polar solvent gives n-butylformamide in quantitative yield.

### 4.3 Production of Formamide (Examples F-1 to F-8)

| | Example F-1 | Example F-2 | Example F-3 | Example F-4 | Example F-5 |
|---|---|---|---|---|---|
| Amine | 48 g of | 44 g of | 50 g of | 60 g of | 58 g of |
| | trihexylamine | trihexylamine | trihexylamine | trihexylamine | trihexylamine |
| Formic acid | 8 g of HCOOH | 5 g of HCOOH | 6 g of HCOOH | 20 g of HCOOH | 10 g of HCOOH |
| Solvent | 35 g of methanol | 41 g of ethanol | 37 g of propanol | — | 29 g of methanol |
| | 10 g of water | 9 g of water | 8 g of water | | |
| Ammonia | 4 g of NH₃ | 3 g of NH₃ | 2 g of NH₃ | 8 g of NH₃ | 5 g of NH₃ |
| Pressure after | 0.18 MPa | 0.11 MPa | 0.10 MPa | 0.89 MPa | 0.27 MPa |
| injection | | | | | |
| Temperature | 160° C. | 160° C. | 160° C. | 160° C. | 160° C. |
| Run time | 4 hours | 4 hours | 4 hours | 4 hours | 4 hours |
| Top phase | 47 g of | 46 g of | 85 g | 56 g of | 57 g of |
| | trihexylamine | trihexylamine | | trihexylamine | trihexylamine |
| Bottom phase | 54 g | 54 g | 14 g | 26 g | 44 g |
| Yield GC | 44.9% | 48.0% | 25.9% | 71.4% | 76.6% |
| (area %) | | | | | |
| formamide | | | | | |
| Yield NMR | 43.0% | 52.0% | 65.0% | not determined | 17.0% |
| (mol %) | | | | | |
| ammonium | | | | | |
| formate | | | | | |
| Selectivity | 78.8% | 100.0% | 74.0% | not determined | 92.3% |
| (yield/conversion) | | | | | |

| | Example F-6 | Example F-7 | Example F-8 |
|---|---|---|---|
| Amine | 60 g of trihexylamine | 57 g of trihexylamine | 16 g of trihexylamine |
| Formic acid | 20 g of HCOOH | 20 g of HCOOH | 3 g of HCOOH |
| Solvent | 20 g of methanol | 20 g of sulfolane | 30 g of 1,4-butanediol |
| Ammonia | 9 g of NH₃ | 8 g of NH₃ | 1 g of NH₃ |
| Pressure after injection | 0.45 MPa | 0.99 MPa | 0.95 MPa |
| Temperature | 160° C. | 160° C. | 160° C. |
| Run time | 4 hours | 4 hours | 4 hours |
| Top phase | 59 g of trihexylamine | 54 g of trihexylamine | 11 g of trihexylamine |
| Bottom phase | 46 g | 45 g | 25 g |
| Yield GC | 72.6% | 89.6% | 6.3% |
| (area %) formamide | | | |
| Yield NMR (mol %) | 26.0% | 10.0% | not determined |
| ammonium formate | | | |
| Selectivity | 98.1% | 99.6% | not determined |
| (yield/conversion) | | | |

The reaction of adducts IIa with ammonia to form formamide gave formamide yields of around 77 area % when carried out in methanol as polar solvent. Ammonium formate present in the reaction mixture at 17 mol % can subsequently be converted into formamide (F-5).

When sulfolane was used as polar solvent, the formamide yield was around 90 area %, including 10 mol % of ammonium formate (F-7).

A deterioration in the yield is observed similarly to the production of dimethylformamide (D-6, D-9) in the presence of methanol plus water (F-1).

Again, water, more preferably water together with primary alcohol as polar solvent, is distillatively removed in process step III-1 and returned as stream 8a into the hydrogenation step.

Specifically in the drawing:

FIG. 1 shows a block diagram of a plant for a preferred process for production of formamides from carbon dioxide by the process of the present invention, and

FIG. 2 shows a block diagram for a more preferred process for production of formamides from carbon dioxide.

In the embodiment according to FIG. 1, carbon dioxide, stream 1, and hydrogen, stream 2, are fed into the hydrogenation reactor I. They are reacted therein in the presence of a catalyst comprising an element from the group 8, 9. or 10 of the periodic table, in the presence of a tertiary amine and in the presence of a polar solvent to form formic acid-amine adducts.

To make good losses of tertiary amine IVa, a replenishment stream of tertiary amine (stream 4a) to the hydrogenation reactor I can be provided.

The two-phase effluent from the hydrogenation reactor I (stream 3) is directed into the phase separation vessel II. The phase separation vessel II contains a bottom phase, enriched with formic acid-amine adducts and also the polar solvent, and a top phase 4, which predominantly comprises the tertiary amine and in which the homogeneous catalyst is present in enriched form, and which is returned from the phase separation vessel II to the hydrogenation reactor I. The bottom phase 5 is fed to an extraction apparatus III in which catalyst residues are extracted with the tertiary amine from the phase separation vessel V (stream 12). The tertiary amine with the catalyst residues (stream 6) from the extraction unit III is returned into the hydrogenation reactor I. The product phase 7 from the extraction unit III is fed to the reactor IV, in which the reaction with ammonia or amines, stream IIIa, takes place. The two-phase liquid reaction effluent, stream 9, from the reactor IV is separated in a phase separation vessel V into a formamide-enriched liquid phase, stream 10, and a second, tertiary amine-enriched liquid phase, stream 12, and stream 12 is preferably recycled into the extraction apparatus III. The stream 10 liquid phase enriched with the target product formamide is distillatively separated in a distillation unit VI to recover the formamide, stream Ia, remove water, H₂O, and unconverted ammonia or amine, stream 11, which is recycled into the reactor IV, and polar solvent (stream 8) which is recycled into the hydrogenation reactor.

The preferred embodiment depicted in FIG. 2 only differs from the embodiment in FIG. 1 in that the extraction unit III connects on its downstream side to a separation unit III-1 to separate off polar solvent. The polar solvent is separated off in the separation unit III-1 and recycled as stream 8a into the hydrogenation reactor I. Reactor IV, where the amidation takes place, can be fed with a solvent other than in the hydrogenation stage, which is separated off again in the distillation unit VI and returned as stream 11 into the amidation, into reactor IV. The product stream 7a from the separation unit III-1 is substantially free of polar solvent, and is forwarded into the reactor IV.

1-9. (canceled)

10. A process for production of formamides of formula Ia 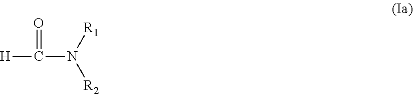
where R₁ and R₂ are each independently selected from the group consisting of hydrogen, linear or branched radicals having from 1 to 15 carbon atoms, cycloaliphatic radicals having from 5 to 10 carbon atoms, a substituted or unsubstituted phenyl radical and a phenylalkyl radical, or R₁ and R₂ are combined to form a five- or six-membered ring comprising an oxygen atom, an N—H radical or an N—R₁ radical, where R₁ is as defined above,
by reacting carbon dioxide with hydrogen in a hydrogenation reactor I in the presence of
a catalyst comprising an element from group 8, 9 or 10 of the periodic table,
a tertiary amine of the formula NR₃R₄R₅ (IVa), where R₃ to R₅ are the same or different and each is independently selected from the group consisting of a branched or unbranched, cyclic or acyclic, aliphatic, araliphatic or aromatic radical having in each case from 1 to 16 carbon atoms, where individual carbon atoms are optionally independently substituted by a hetero group selected from the group consisting of —O— and >N—, and also two or all three radicals may be linked together to form a chain comprising at least four atoms in each case, with the proviso that the total number of carbon atoms is at least 6, and also
a polar solvent
to form formic acid-amine adducts as intermediates of formula IIa
NR₃R₄R₅.xᵢHCOOH (IIa),
(xᵢ=0.4 to 5)
wherein two liquid phases form in said hydrogenation reactor I in the course of the reaction, viz., a bottom phase comprising predominantly the polar solvent, in which the formic acid-amine adducts are enriched, and also a top phase comprising predominantly the tertiary amine, in which the homogeneous catalyst is enriched, and wherein
the effluent from said hydrogenation reactor I is worked up in accordance with the following process steps:
separating the two liquid phases from said hydrogenation reactor I in a phase separation vessel II,
returning said top phase from said phase separation vessel II into said hydrogenation reactor I, and
forwarding said bottom phase from said phase separation vessel (II) into an extraction unit III, in which
residues of catalysts are extracted with the same tertiary amine as used in the hydrogenation and the tertiary amine laden with catalyst is recycled into said hydrogenation reactor I,
which comprises
(i) forwarding the catalyst-free stream comprising the formic acid-amine adducts of formula (Ha) and polar solvent into a reactor IV, and therein reacting the formic acid-amine adducts of formula (IIa) at temperatures of 0 to 200° C. with ammonia or amines of formula (Ia), 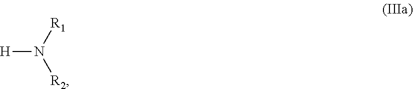
where R₁ and R₂ are each as defined under formula (Ia),
to obtain via the interstage having formula (Va) of the corresponding adduct of formic acid with ammonia or an amine of formula (IIIa)
NHR₁R₂.xHCOOH (Va)
the corresponding formamide of formula (Ia), and
(ii) separating the two-phase liquid reaction effluent from said reactor IV (stream 9) in a phase separation vessel V into a liquid phase (stream 10) enriched with formamides of formula (Ia) and a second liquid phase (stream 12) enriched with tertiary amine and wholly or partly returning the liquid phase (stream 12) enriched with tertiary amine into said extraction unit III, and
(iii) separating stream 10 in a distillation unit VI by distillation to recover said formamide of formula (Ia), remove water from the system, return the polar solvent (stream 8) into said hydrogenation reactor I and return the unconverted ammonia or amine of formula (IIIa) into said reactor IV (stream 11).

11. The process according to claim 10, wherein the part stream (stream 12) (liquid phase enriched with tertiary amine) that is not returned into said extraction unit III is returned into said hydrogenation reactor I.

12. The process according to claim 10, wherein the polar solvent is removed from stream 7 (laden with formic acid-amine adducts of formula (Ia)) before said stream 7 is fed to said reactor IV.

13. The process according to claim 10, wherein the polar solvent used is methanol, ethanol, propanol, butanol, formic ester of the abovementioned alcohols, water or a mixture thereof.

14. The process according to claim 10, wherein the polar solvent is a diol its formic ester.

15. The process according to claim 10, wherein the polar solvent is 1,3-propanediol, 2-methyl-1,3-propanediol or 1,4-butanediol or their formic esters.

16. The process according to claim 10, wherein the polar solvent used comprises sulfone, sulfoxide, open-chain or cyclic amide or mixtures thereof.

16. The process according to claim 10, wherein the returning of said top phase from said phase separation vessel II into said hydrogenation reactor I is effected at an overall pressure in the range from 0.2 to 30 MPa and a temperature in the range from 20 to 200° C.

17. The process according to claim 10, wherein the pressure in said hydrogenation reactor I and in said phase separation vessel II is the same or substantially the same.

18. The process according to claim 10, wherein the temperature in said hydrogenation reactor I and in said phase separation vessel II is the same or substantially the same.

19. The process according to claim 10, wherein the tertiary amine of the formula NR₃R₄R₅ (IVa), where R₃ to R₅ are the same or different and each is independently selected from the group consisting of a branched or unbranched, cyclic or acyclic, aliphatic, araliphatic or aromatic radical having in each case from 1 to 12 carbon atoms.

20. The process according to claim 11, wherein the polar solvent is removed from stream 7 (laden with formic acid-amine adducts of formula (Ia)) before said stream 7 is fed to said reactor IV.

21. The process according to claim 20, wherein the returning of said top phase from said phase separation vessel II into said hydrogenation reactor I is effected at an overall pressure in the range from 0.2 to 30 MPa and a temperature in the range from 20 to 200° C.

22. The process according to claim 21, wherein the pressure in said hydrogenation reactor I and in said phase separation vessel II is the same or substantially the same.

23. The process according to claim 22, wherein the temperature in said hydrogenation reactor I and in said phase separation vessel II is the same or substantially the same.

24. The process according to claim 23, wherein the polar solvent used is methanol, ethanol, propanol, butanol, formic ester of the abovementioned alcohols, water or a mixture thereof.

25. The process according to claim 23, wherein the polar solvent is 1,3-propanediol, 2-methyl-1,3-propanediol or 1,4-butanediol or their formic esters.

26. The process according to claim 23, wherein the polar solvent used comprises sulfone, sulfoxide, open-chain or cyclic amide or mixtures thereof.
